(19) United States
(12) Patent Application Publication
(10) Pub. No.: US 2013/0053556 A1
(43) Pub. Date: Feb. 28, 2013

(54) Photocleavable morpholino oligos with integral photolinkers for modulating the activity of any selected gene transcript by exposure to light

(71) Applicants: Yong-Fu Li, Corvallis, OR (US); James E. Summerton, Corvallis, OR (US); Paul A. Morcos, Corvallis, OR (US)

(72) Inventors: Yong-Fu Li, Corvallis, OR (US); James E. Summerton, Corvallis, OR (US); Paul A. Morcos, Corvallis, OR (US)

(73) Assignee: GENE TOOLS, LLC, Philomath, OR (US)

(21) Appl. No.: 13/199,337

(22) Filed: Aug. 26, 2011

(51) Int. Cl. C07F 9/6561; C07D 233/61

(52) U.S. Cl. 544/81; 548/334.1

(57) ABSTRACT

Morpholinos are widely used to block the activity of selected single-stranded genetic sequences. This invention comprises Morpholinos containing one or more integral photolinkers (Photo-Morpholinos) wherein the photolinkers are directly incorporated into the sequence of a Morpholino, where the photolinker has a size and structure which emulates the size and structure of a Morpholino subunit. This integrated photolinker design substantially simplifies and reduces cost of production relative to earlier photocleavable compositions.

The invention also comprises use of these Photo-Morpholinos for modulating the expression of any selected gene transcript at any selected time and at any selected site simply by exposure to light. These Photo-Morpholinos afford a new use wherein a gene transcript is rendered inactive by contacting with a Photo-Morpholino—and then later exposure to light cleaves the Photo-Morpholino to inactive fragments—thereby reactivating that previously inactivated gene transcript.

### FIELD OF THE INVENTION

This invention is directed to compositions and methods for modulating the expression of any selected gene transcript at any selected time and at any selected site by a brief exposure to light.

### BACKGROUND OF THE INVENTION

A prerequisite for a continuing improvement in understanding of processes in single cells and in organisms is a constant improvement in the available experimental tools. An important general criterion is, for example, how selectively a particular aspect in a cell or organism can be manipulated. To achieve spatial and/or temporal control one strategy is to put the compound under the control of a conditional trigger which can be either internal or external. Light is an ideal external trigger because in most cases animal cells do not react to light (except highly specialized cells, such as the photoreceptors of the eye). Also, the wavelengths used can be long enough that the cells are not harmed by the light. In addition, most of the cells which are commonly studied in laboratories are transparent and the same is true for many small model organisms, such as the zebrafish, the nematode and the Xenopus which can be transparent throughout early development or even longer for certain organisms.

Morpholino oligos are the most commonly used antisense technology for gene regulation. The success of these Morpholinos lies in their general lack of cellular toxicity, their high efficacy and specificity, their simplicity of application, and their complete stability in biological systems. Once the sequence of a gene is known, a Morpholino oligo can be designed, synthesized and administered, and gene function can be determined by examining the resultant phenotype or by appropriate biochemical analysis. However, Morpholino-mediated gene regulation has been limited by a lack of temporal and spatial control. This can be a particular problem when modulation of gene function produces an early lethal phenotype, precluding analysis of gene function later in development.

Previous efforts to generate conditionally active Morpholinos have focused mainly on using a photocleavable leash or a photocleavable RNA strand to mask the Morpholino oligo. For example, Chen et al (Nature Chemical Biology 3:650-651(2007), and U.S. Pat. No. 7,923,562) recently described the synthesis of a Morpholino oligo joined through a photocleavable leash to a short masking Morpholino oligo. Upon photolysis the leash was cleaved, which allowed the short Morpholino to dissociate from the long Morpholino, which in turn allowed the long Morpholino to bind and block expression of its complementary target RNA. In addition, Mayer et al. (Genesis 47:736-743 (2009)) utilized a conventional Morpholino oligo which was blocked by a complementary strand of RNA containing a photocleavable link in its center. Exposure of the Morpholino/RNA duplex to light cleaved the RNA, which thereby released the Morpholino to bind and block expression of its complementary target RNA.

While these earlier efforts have provided proof of principle for this general light-mediated modulation of gene expression, these prior conditionally-active Morpholinos are quite complicated and expensive to produce, and so are unduly costly and so poorly suited as commercial products. Thus, there currently is no reported photocleavable Morpholino technology which appears well suited for commercial production.

To address this need for a cost-effective photocleavable Morpholino technology, photolinkers have been designed to replace a Morpholino subunit in the course of Morpholino oligo assembly. This allows for the first time direct incorporation of an integral photolinker into a Morpholino oligo—thereby affording routine cost-effective automated synthesis of photocleavable Morpholino oligos (Photo-Morpholinos) that are expected to serve as a new class of affordable research reagents suitable for routine spatio-temporal control of gene expression in transparent biological systems.

These Photo-Morpholinos can be readily structured to offer either of two modes of action.
a) The “light-off” mode entails pairing a Photo-Morpholino with a conventional Morpholino oligo to give an inactive duplex which is introduced into the organism. Then at a selected time light is used to cleave the Photo-Morpholino strand of the duplex, thereby unmasking the conventional Morpholino oligo—which then acts to turn off the expression of its targeted gene transcript.
b) A new “light-on” mode entails delivering a Photo-Morpholino to turn off its targeted gene transcript, and then at a selected time light is used to cleave that previously-delivered Photo-Morpholino, thereby turning back on the expression of its targeted gene transcript.

### SUMMARY OF THE INVENTION

The structures of photolinkers of the invention are disclosed herein and illustrated in FIG. 1. Also disclosed herein, and illustrated in FIG. 2, is a method for direct incorporation of a photolinker during assembly of a Morpholino oligo to generate a Photo-Morpholino of the invention containing one or more integral photolinkers. The chemical transformations leading to photocleavage of the Photo-Morpholino is also described herein, and is illustrated in FIG. 3.

Design and use of Photo-Morpholinos in the “light-on” mode is illustrated in FIG. 4.

Design and use of Photo-Morpholinos in the “light-off” mode is illustrated in FIG. 5.

The photolinker is a bifunctional structure suitable for replacement of a Morpholino subunit in the oligo assembly process. The photolinker has an electrophilic moiety which serves to acylate the N-terminus of a growing Morpholino oligo, and the photolinker also has a protected amine moiety which, after deprotection, is used for continued elongation of the Photo-Morpholino after insertion of the photolinker. In some embodiments the light-sensitive component of the photolinker is a nitrobenzyl moiety. In other embodiments the light-sensitive component is a mono-methoxy or dimethoxy nitrobenzyl moiety. As noted, after incorporation the integral photolinker replaces a subunit in the Morpholino oligo. The size of the photolinker is designed to closely match the spacing of a Morpholino subunit in terms of bond distance, so that the interaction between an antisense Photo-Morpholino and the complementary sense RNA, or between a sense Photo-Morpholino and its complementary antisense conventional Morpholino will be only minimally compromised due to the replacement of a conventional Morpholino subunit by the photolinker.

“Light-On” Application

One application of a Photo-Morpholino entails initial blockage of the expression of a selected RNA transcript, and then subsequent exposure to light to effect unblocking of that RNA transcript. More specifically, as illustrated in FIG. 4, an antisense Photo-Morpholino is contacted with its complementary target RNA transcript, which acts to block the expression of that RNA transcript. Subsequently, at a selected time the blocked transcript is briefly exposed to light in order to cleave the RNA-bound Photo-Morpholino into inactive fragments which then release from the RNA transcript. As a consequence of that light exposure, the RNA transcript is unblocked and so can resume its normal functions, such as splicing, transport, and translation.

“Light-Off” Application

The other principal application of a Photo-Morpholino entails introduction into a biological system a conventional Morpholino which is masked by a complementary Photo-Morpholino to give an inactive duplex. Subsequently, at a selected time the inactive duplex is exposed to light in order to cleave the Photo-Morpholino into inactive fragments, which release from the conventional Morpholino. The conventional Morpholino then acts to bind and block its complementary RNA transcript, thereby blocking its normal functions, such as splicing, transport, and translation.

Therefore, the invention allows one to use brief exposure to light to either turn on the normal activities of its targeted RNA transcript, or turn off the normal activities of its targeted RNA transcript. Further, in addition to temporal control by virtue of when the light exposure occurs, one can also exert spatial control by using a narrow beam of light to selectively expose a specific area—resulting in turning on or turning off targeted RNA transcripts in just that selected area.

It should also be appreciated that these same “light-on” and “light-off” strategies for light-mediated modulation of the activity of RNA transcripts are suitable for use with a wide variety of RNA transcript types, including: pre-messenger RNAs, messenger RNAs, transfer RNAs, micro RNAs, short interfering RNAs, ribozymes, ribosomal RNAs, and viral genomic RNAs.

### BRIEF DESCRIPTION OF THE DRAWINGS

FIGS. 1(a) and (b) illustrate the synthesis of a single-cleavage and a dual-cleavage photolinker.

FIGS. 2(a) and (b) illustrate the incorporation of a photolinker into a Photo-Morpholino.

FIGS. 3(a) and (b) show light-induced cleavage of an integral photolinker of a Photo-Morpholino.

FIGS. 4(a) and (b) illustrate several “light-on” applications of Photo-Morpholinos.

FIGS. 5(a) through (d) illustrate several “light-off” applications of Photo-Morpholinos.

### DETAILED DESCRIPTION OF THE INVENTION

I. Abbreviations and Definitions

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

Morpholino Subunit: A Morpholino subunit has a standard nucleic acid base which is bound to morpholine ring, as described in Summerton & Weller, Antisense & Nucleic Acid Drug Development 7: 187-195 (1997).

Morpholino: A Morpholino oligo having a sequence of Morpholino subunits linked via non-ionic phosphorodiamidate groups to give a linear chain having a defined sequence of Morpholino subunits.

Antisense Morpholino: An Antisense Morpholino contains a defined sequence of nucleobases which is complementary to a corresponding number of bases in its targeted RNA transcript.

Sense Morpholino: A Sense-Morpholino is complementary to its antisense Morpholino.

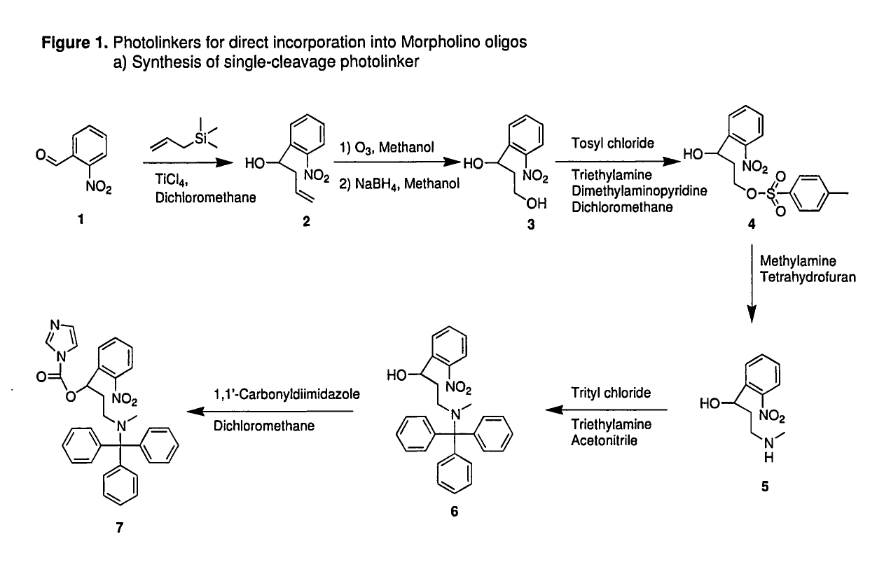

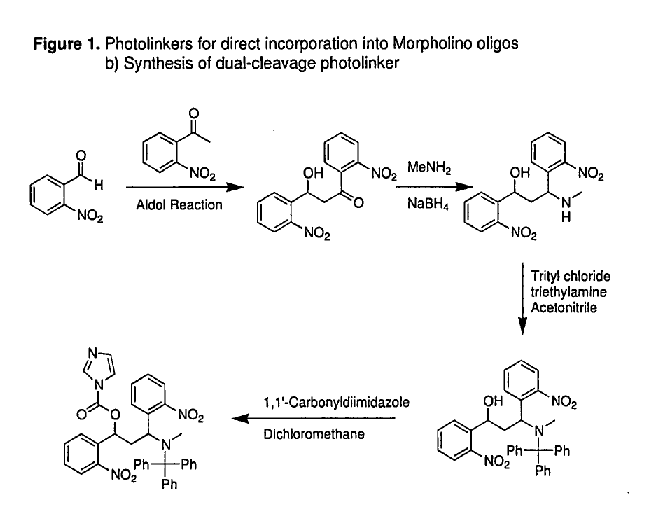

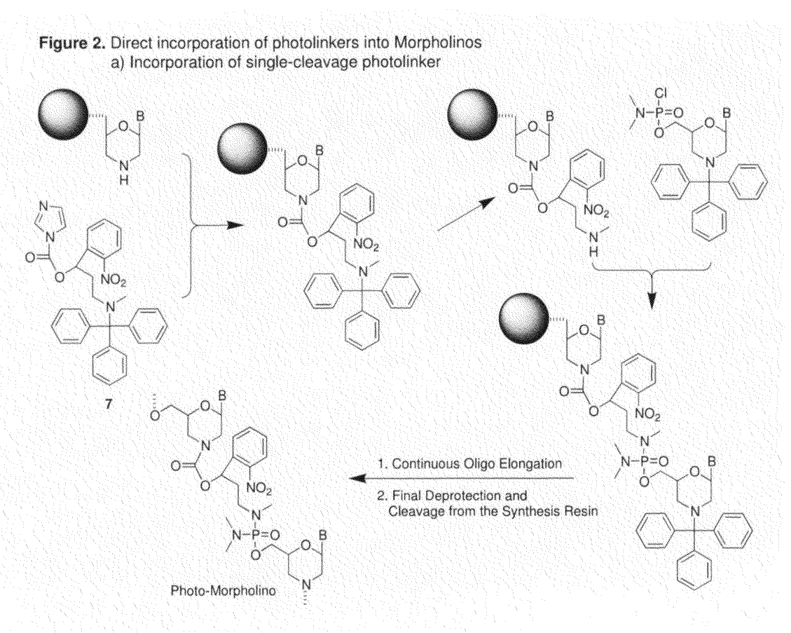

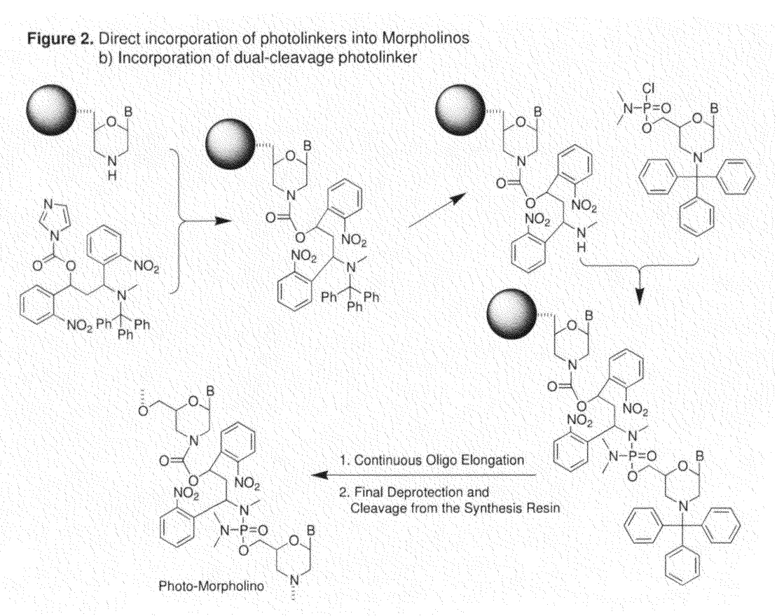

Photolinker. A photocleavable linker having a structure shown in FIG. 1 or FIG. 2.

Photo-Morpholino: A Morpholino which contains one or more integral photolinkers.

Light: In this invention UV light is used for the photocleavage step which has a wavelength in the range of about 320 nm to 420 nm, and preferably in the range of 340 nm to 390 nm.

II. General

Morpholinos are often used as research tools for blocking the function or modifying splicing of any selected gene transcripts, and are also in development as therapeutics targeted against pathogenic organisms, and for amelioration of genetic diseases. Because of their novel backbone structure, Morpholinos are not recognized by degradative enzymes, and so are not cleaved by nucleases in cells or in serum. Activities of Morpholinos against a variety of RNA transcripts, including mRNAs, microRNAs, and ribozymes, demonstrate that they can be used as a general-purpose tool for blocking interactions of proteins or nucleic acids with selected sites in RNA transcripts.

The use of Morpholino antisense oligos to regulate gene expression is therefore of great interest. In many cases, modulation of gene function with Morpholino oligos have been achieved primarily through micro-injection into zebrafish or frog eggs, after which the activity of the targeted RNAs continue to be modulated for multiple days. However, this does not permit conditional modulation of the RNA transcripts. One approach for overcoming temporal and/or spatial limitations on modulation of the function of RNA transcripts utilizes photocleavable constructs, thereby providing for temporal and/or spatial specificity.

III. Compositions

The present invention relates to the design and synthesis of photolinkers and incorporation of those photolinkers into Morpholinos to give Photo-Morpholinos containing one or more integral photolinkers. Such photocleavable linkers were designed to closely match the spacing of a Morpholino subunit between oxygen and nitrogen atoms in terms of bond distance (see the figure below).

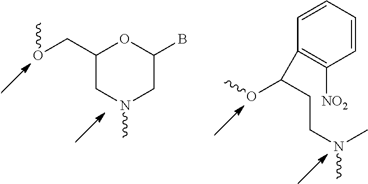

With this photolinker design, its incorporation into the Morpholino causes only minimal impact on the structure of the Morpholino oligo, which is beneficial for the effective binding of the Photo-Morpholino to its complementary RNA (for light-on applications) or complementary Morpholino strand (for light-off applications).

The structure of the photolinker allows routine incorporation in the course of Morpholino assembly, as illustrated in FIG. 2. The photolinkers of the invention are selected from the structures:

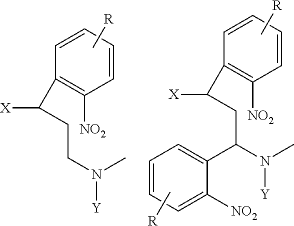

R is one or more substituents on the phenyl ring. This includes, without limitation: hydrogen atoms, mono-methoxy or di-methoxy groups.

Y is a protecting group selected from commonly used amino protecting groups such as: trityl, methyltrityl or methoxytrityl, or the FMOC group.

X is a reactive moiety effective to form a covalent bond with the nitrogen atom of a Morpholino subunit. This includes, without limitation: a reactive acylating agent, such as:

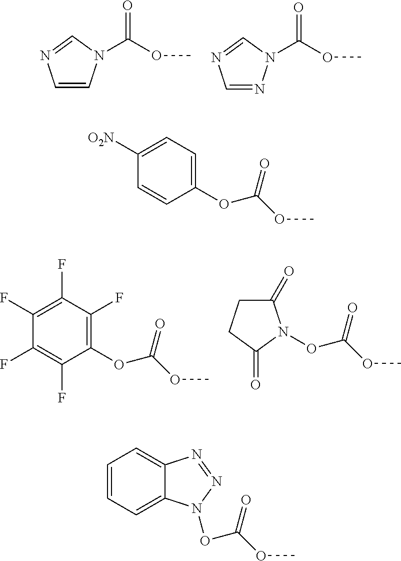

Generally, one photolinker is appropriate for insertion into a Morpholino of a length between 12 and 25 subunits, and two photolinkers are appropriate for insertion into a Morpholino of a length between about 23 and 38 subunits. In the case of a single photolinker, preferably the photolinker is placed at or near the center of the Photo-Morpholino chain. In the case of two photolinkers, they are distributed at about ⅓ and about ⅔ of the way along the Morpholino chain. A section of a conventional Morpholino (left side) and a representative section of a Photo-Morpholino (right side) are shown below:

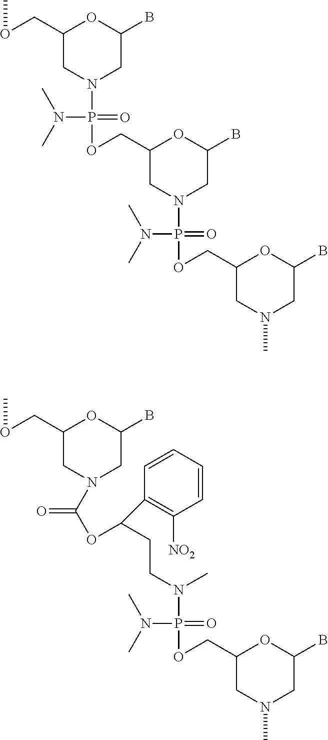

As can be seen in the above structures, the photolinker of the invention provides a rather close match to the spacing of the normal Morpholino subunit which the photolinker replaces—thereby causing only minimal perturbation of pairing of the Photo-Morpholino to its complementary strand.

IV. Synthesis of Photolinkers

a) Photolinker with a Single Cleavage Site

FIG. 1a illustrates the synthesis of a single-cleavage photolinker using 2-nitrobenzaldehyde (1) as the starting material. Allylation of the aldehyde 1 proceeds with allyltrimethylsilane in the presence of titanium (IV) chloride at low temperature to give a secondary alcohol 2. The double bond of 2 is cleaved by ozonolysis to generate an aldehyde intermediate which is reduced in situ by sodium borohydride to the corresponding alcohol 3. Selective sulfonation of the primary alcohol is achieved by treatment with tosyl chloride in the presence of triethylamine and N,N-dimethylaminopyridine to give sulfonate 4. Methylamine derivative 5 is obtained by exposing the sulfonate 4 with methylamine solution. The amine 5 is then protected with trityl group to give the alcohol intermediate 6 which is activated by 1, 1′-carbonyldiimidazole to afford the activated photocleavable linker 7.

b) Photolinker with Dual Cleavage Sites

FIG. 1b illustrates the synthesis of a dual-cleavage photolinker using 2-nitrobenzaldehyde as the starting material. Aldol condensation with 2′-nitroacetophenone gives beta-hydroxyketone which undergoes subsequent reductive amination with methylamine to furnish a secondary amine. Protection of the secondary amine with trityl group, followed by the activation of the secondary alcohol with 1,1′-carbonyldiimidazole affords the activated dual cleavage photolinker.

V. Incorporation of Photolinker During Morpholino Assembly

FIG. 2 illustrates the synthesis of a Photo-Morpholino on a solid phase synthesis resin. During the Morpholino assembly process wherein the growing Morpholino chain is ready for further coupling, the photolinker (either single-cleavage photolinker (FIG. 2a) or dual-cleavage photolinker (FIG. 2b)) is added instead of a Morpholino subunit and coupling is carried out under conditions used for coupling of a Morpholino subunit. After the photolinker is coupled to the nascent Morpholino on the synthesis resin, the trityl group of the photolinker is removed—which generates the secondary amine required for addition of subsequent Morpholino subunits to the nascent chain. If desired, a photolinker can be inserted at two or more sites in the Photo-Morpholino chain.

Final cleavage of the oligo from the synthesis resin and concomitant deprotection of the nucleobases gives the desired Photo-Morpholino. The whole assembly process is readily automated for streamlined parallel assembly of multiple Photo-Morpholinos. This ability to integrate one or more photolinkers into multiple Morpholinos in parallel in a single automated synthesis run constitutes a valuable and cost-effective advantage over prior art production procedures for photocleavable Morpholinos.

VI. Application of Photo-Morpholinos for Gene Regulation

a) Cleavage by Light

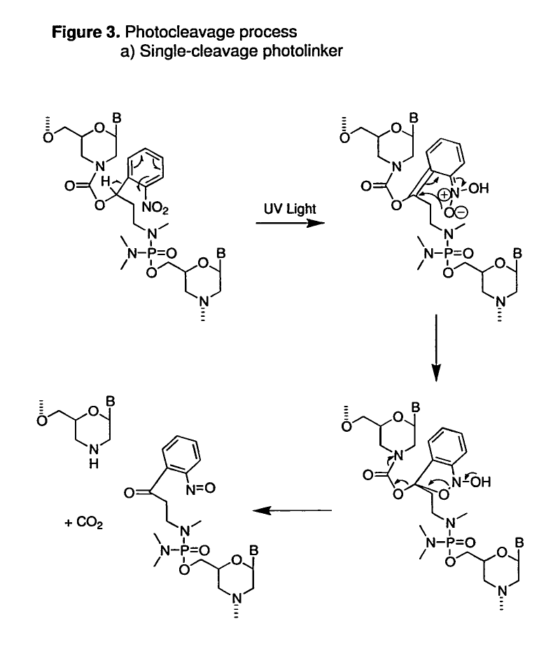

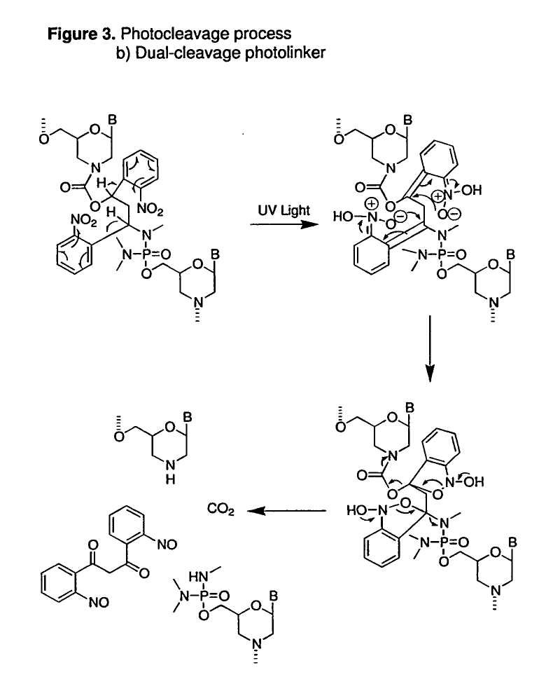

FIG. 3 illustrates the mechanism by which Photo-Morpholinos are cleaved by light. Under light irradiation, normally at about 365 nm wavelength, the aci-nitro intermediate is generated via n→π excitation and intramolecular hydrogen atom transfer from the alfa-carbon of the secondary alcohol. The aci-nitro intermediate cyclizes to form the heterocyclic five membered ring, and subsequently collapses to result in the elimination of carbon dioxide, generating one fragment of the Morpholino. For single cleavage photolinker, the other fragment retains the resultant nitroso and ketone components (FIG. 3a). For dual-cleavage photolinker, the cleavage occurs at both sites, releasing Morpholino fragments and 1,3-di(2-nitrosophenyl)-1,3-propanedione (FIG. 3b).

b) “Light-On” Applications

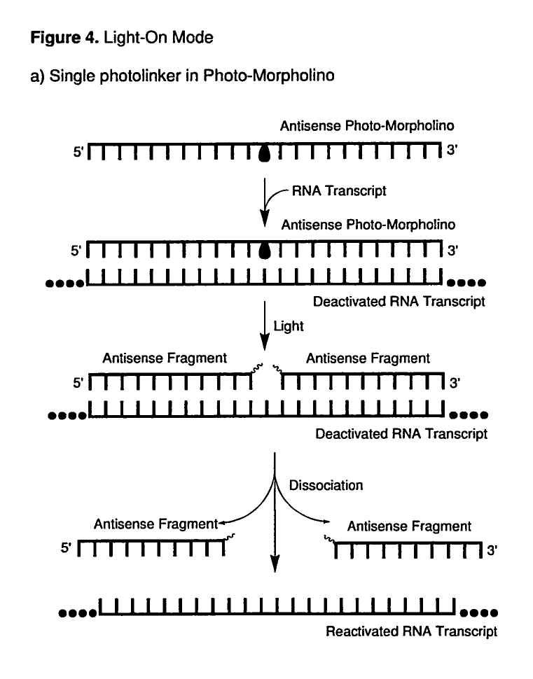

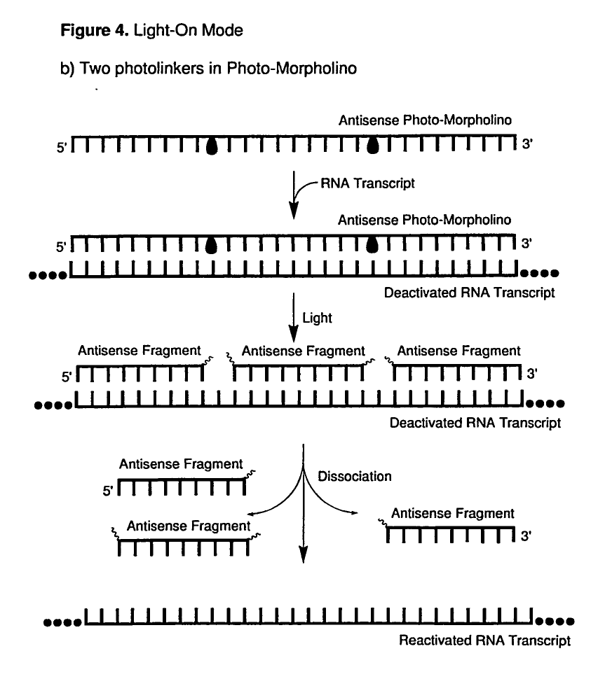

The first of the two principal applications of Photo-Morpholinos entails using light to turn on the expression of selected gene transcripts, as illustrated in FIG. 4. This entails initially contacting in a biological system the targeted RNA transcript with a complementary antisense Photo-Morpholino. This serves to block the expression of that RNA transcript. Subsequently, at a selected time that blocked RNA transcript is briefly exposed to light, which cleaves the RNA-bound Photo-Morpholino into inactive fragments which then release from the RNA transcript. As a consequence of that light exposure, the RNA transcript is unblocked and so can resume its normal functions, such as splicing, transport, and translation. Thus, exposure to light serves to turn back on the normal biological expression of the selected gene transcript or target sequence within that transcript. Use of only a narrow beam of light for the exposure extends the above temporal control to include spatial control as well.

c) “Light-Off” Applications

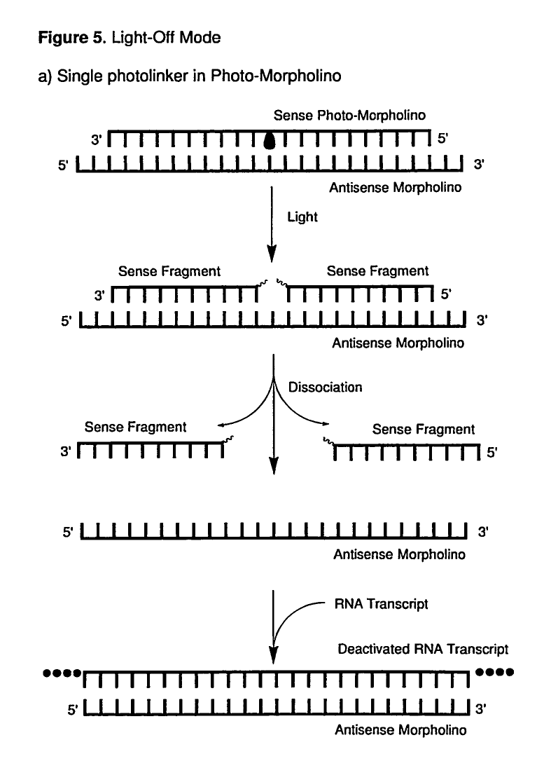

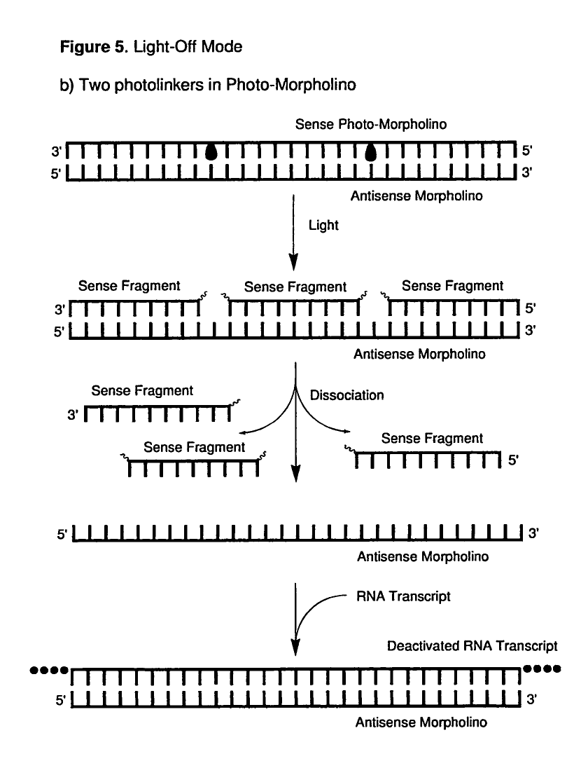

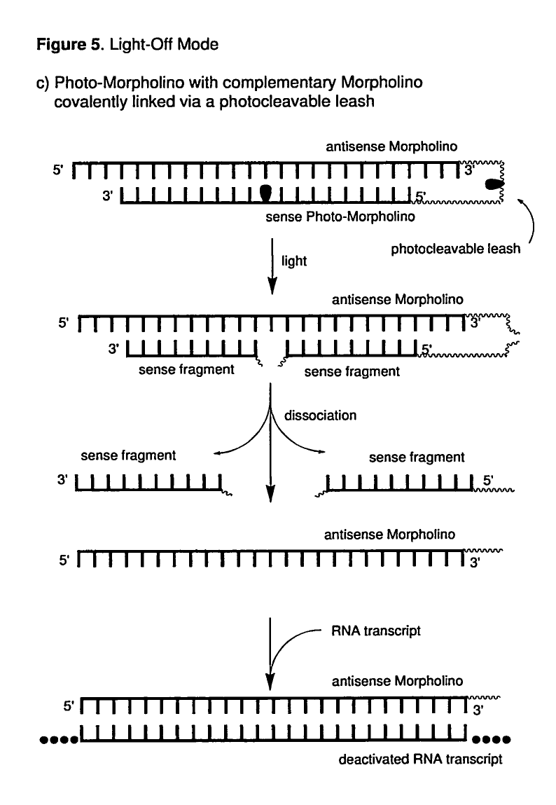

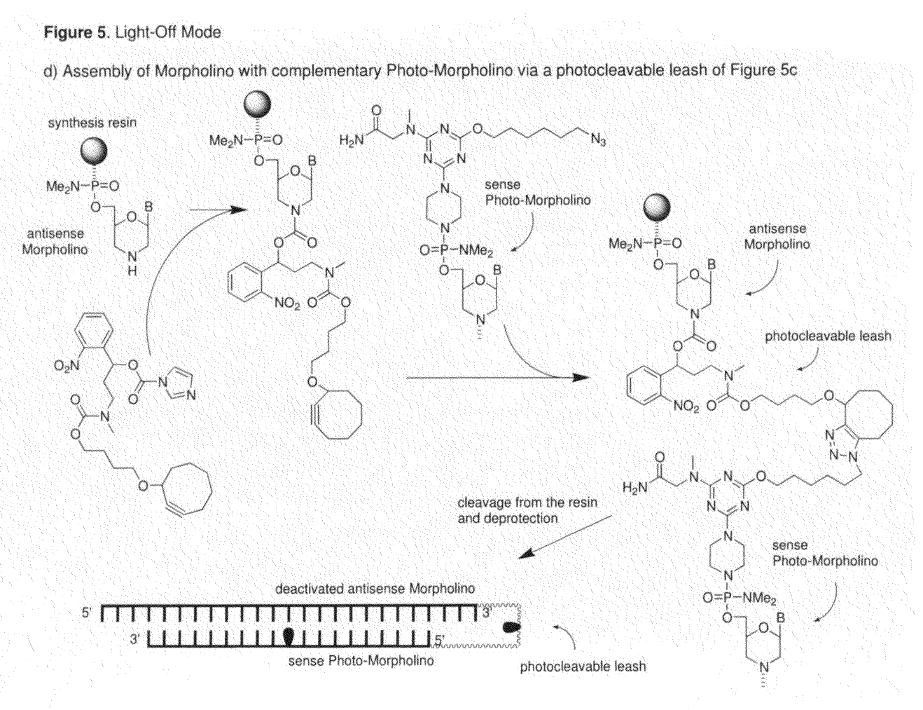

The second of the two principal applications of Photo-Morpholinos entails using light to turn off the expression of selected gene transcripts, as illustrated in FIG. 5. This entails introduction into a biological system a conventional antisense Morpholino which is paired with a complementary sense Photo-Morpholino to give an inactive duplex. Subsequently, at a selected time the inactive duplex is exposed to light in order to cleave the Photo-Morpholino into inactive fragments, which release from the conventional antisense Morpholino. The conventional antisense Morpholino then acts to bind and block its complementary RNA transcript, thereby blocking its normal functions, such as splicing, transport, and translation. Thus, exposure to light serves to turn off the normal biological expression of the selected gene transcript or target sequence within that transcript. Use of only a narrow beam of light for the exposure extends the above temporal control to include spatial control as well.

VII. Test System for Optimizing Photo-Morpholinos

While prior art photocleavable antisense systems have typically been developed and optimized using zebrafish embryos, such test systems are complicated, time consuming, difficult to quantitate, and exhibit high variability. Instead, we recommend for initial studies the use of a coupled transcription/translation in vitro test system which affords fast, simple, inexpensive assays of RNA transcript activities in both the light-on and light-off experiments with Photo-Morpholinos. The “TnT T7 Quick Coupled Transcription/Translation System” from Promega Corporation is particularly suitable for this purpose. Such a test system provides a highly quantitative readout of light emitted by the luciferase which is coded by the RNA transcript generated in the system.

Light-On Experiments (See FIG. 4)

A concentration of between 200 nM and 400 nM for the antisense Photo-Morpholino is generally suitable for the antisense Photo-Morpholino concentration.

For Photo-Morpholinos containing one photolinker (see FIG. 4a) our preliminary results suggest the Photo-Morpholino should have a length from about 16 to about 28 Morpholino subunits. However, the optimal length for the Photo-Morpholino is dependent on the particular target sequence in the selected RNA transcript, and appears to be substantially dependent on the G+C content and the specific sequence of the two antisense fragments generated by the photocleavage step. It should be noted that improvement in activity can sometimes be achieved by moving the site at which the photolinker is integrated in the Photo-Morpholino in order to better match the binding affinities of the two antisense fragments generated in the photocleavage step. Studies are now in progress for optimizing target selection criteria.

For Photo-Morpholinos containing two photolinkers (see FIG. 4b) our preliminary results suggest the Photo-Morpholino should have a length from about 21 to about 33 Morpholino subunits. Again, the optimal length for the Photo-Morpholino is dependent on the particular target sequence in the selected RNA transcript, and appears to be substantially dependent on the G+C content and the specific sequence of the three antisense fragments generated by the photocleavage step.

Light-Off Experiments (See FIG. 5)

A concentration of between 200 nM and 400 nM for the antisense Morpholino is generally a suitable concentration for these experiments. The principal challenge in obtaining good results in the light-off applications is identifying the best length for the sense Photo-Morpholino. In essence, the challenge is to select a length wherein the full-length sense Photo-Morpholino has a high affinity for its complementary antisense Morpholino, while the sense Morpholino fragments generated by exposure to light have only a minimal affinity for the complementary antisense Morpholino.

For Photo-Morpholinos containing one photolinker (see FIG. 5a) our preliminary results suggest the Photo-Morpholino should have a length from about 14 to about 26 Morpholino subunits. However, again the optimal length for the Photo-Morpholino is dependent on the G+C content and the specific sequence of the two sense fragments generated by the photocleavage step. Again, improvement in activity can sometimes be achieved by moving the site at which the photolinker is integrated in the Photo-Morpholino in order to better match the binding affinities of the two sense fragments for their complementary antisense Morpholino.

For Photo-Morpholinos containing two photolinkers (see FIG. 5b) our preliminary results suggest the Photo-Morpholino should have a length from about 21 to about 36 Morpholino subunits. However, again the optimal length for the Photo-Morpholino is dependent on the G+C content and the specific sequence of the three sense fragments generated by the photocleavage step. Again, improvement in activity can sometimes be achieved by moving the site at which the photolinkers are integrated in the Photo-Morpholino in order to better match the binding affinities of the three sense fragments for their complementary antisense Morpholino.

For Photo-Morpholinos containing one photolinker and a photocleavable leash (see FIGS. 5c and 5d) the intact leash enhances blockage of the antisense Morpholino prior to exposure to light without enhancing the affinity of the cleavage fragments for the antisense Morpholino. In this particular case the sense Photo-Morpholino can be even shorter than in the case shown in FIG. 5a. In this combination scheme utilizing a photocleavable leash and an integrated photolinker the Photo-Morpholino should have a length from about 12 to about 24 Morpholino subunits, again depending on G+C content and sequence.

### EXAMPLES

The practice of the present invention will employ, unless otherwise indicated, conventional techniques of synthetic organic chemistry, biochemistry and the like, which are within the skill of art. The following examples are put forth so as to provide those of ordinary skill in the art with a complete disclosure and description of how to make and use the compositions/compounds/methods of the invention. All components were obtained commercially unless otherwise indicated.

### Example 1

### Synthesis of 1-(2-nitrophenyl)-but-3-en-1-ol (2)

To a stirred solution of 2-nitrobenzaldehyde (1) (5.956 g, 39.41 mmol) in dry dichloromethane (100 ml) was added TiCl₄ (100 ml of 1M solution in dichloromethane, 100 mmol) and allyltrimethylsilane (14.91 ml, 93.41 mmol) at −78° C. After the reaction mixture was stirred for 30 min, it was washed twice with 1N HCl (150 ml each) and once with water (150 ml). After drying over anhydrous sodium sulfate, most of the solvent was removed in vacuo, the residue was purified by silica gel chromatography (dichloromethane as solvent for elution) to give yellowish oil (7.0 g, 92%).

¹H NMR (400 MHz, CDCl₃), δ=2.40-2.48 (2H, m), 2.70-2.76 (1H, m), 5.20 (1H, m), 5.24 (1H, m), 5.34 (1H, br-d, J=7.32 Hz), 5.86-5.97 (1H, m), 7.44 (1H, ddd, J=1.36, 8.57, 9.27 Hz), 7.67 (1H, ddd, J=1.16, 7.28, 8.55 Hz), 7.85 (1H, dd, J=1.28, 7.90 Hz), 7.95 (1H, dd, J=1.18, 8.12 Hz).

### Example 2

### Synthesis of 1-(2-nitrophenyl)-propane-1, 3-diol (3)

A solution of 1-(2-nitrophenyl)-but-3-en-1-ol (2) (7 g, 36.23 mmol) in methanol (250 ml) at −78° C. was saturated with ozone for 5 min, producing a blue-colored reaction mixture. Sodium borohydride (7.4 g, 195 mmol) was added to the solution at −78° C., and the reaction mixture allowed to warm to room temperature over a period of 30 min. The reaction mixture was quenched with saturated aqueous ammonium chloride (160 ml). Methanol was removed in vacuo, and the aqueous layer was extracted with ethyl acetate (250 ml). Solvent was removed in vacuo, and the residue was purified by silica gel chromatography to give a colorless liquid (4.9 g, 69%).

¹H NMR (400 MHz, CDCl₃), δ=1.94-2.03 (1H, m), 2.09-2.15 (1H, m), 2.47 (1H, br-s), 3.71 (1H, d, J=2.78 Hz), 3.93-4.03 (2H, m), 5.52 (1H, m), 7.44 (1H, ddd, J=1.39, 8.37, 8.37 Hz), 7.68 (1H, ddd, J=1.18, 7.85, 8.52 Hz), 7.92 (1H, dd, J=1.26, 8.04 Hz), 7.95 (1H, dd, J=1.01, 8.25 Hz).

### Example 3

### Synthesis of 3-(2-nitrophenyl)-3-hydroxy-propyl toluene-4-sulfonate (4)

Tosyl chloride (2.29 g, 12 mmol) was added to a mixture containing 1-(2-nitrophenyl)-propane-1,3-diol (3) (2.366 g, 12 mmol), triethylamine (5.02 ml, 36 mmol) and N,N-dimethylaminopyridine (2.20 g, 18 mmol) in dichloromethane (60 ml) cooled in an ice-bath. The ice-bath was removed after addition of the reagents. The reaction mixture was kept at room temperature for 1 hour. The volatile reagents were removed by evaporation. The residue was chromatographed on a silica gel column to give yellowish oil (2.2 g, 52%).

¹H NMR (400 MHz, CDCl₃), δ=2.01-2.10 (1H, m), 2.21-2.29 (1H, m), 2.48 (3H, s), 2.55 (1H, br-s), 4.25-4.30 (1H, m), 4.38-4.44 (1H, m), 5.32-5.36 (1H, m), 7.38 (2H, d, J=8.07 Hz), 7.46 (1H, ddd, J=1.45, 8.41, 8.41 Hz), 7.66 (1H, ddd, J=0.81, 7.76, 7.76 Hz), 7.79 (1H, dd, J=0.27, 7.74 Hz), 7.84 (2H, d, J=8.40 Hz), 7.95 (1H, dd, J=0.95, 8.32 Hz).

### Example 4

### Synthesis of 1-(2-nitrophenyl)-3-(N-methyl-N-tritylamino)propanol (6)

A solution of 3-(2-nitrophenyl)-3-hydroxy-propyl toluene-4-sulfonate (4) (2.4 g, 6.83 mmol) and methylamine (40 ml of 2.0 M solution in THF, 80 mmol) was stirred at room temperature for 3 days. The solvent was removed in vacuo, co-evaporated twice with triethylamine. The crude amine 5 was then dissolved in acetonitrile (50 ml). To the mixture was added triethylamine (3.8 ml, 27.3 mmol), followed by tritylchloride (2 g, 7.17 mmol). The mixture was kept at room temperature for 30 min. The reaction mixture was diluted with ethyl acetate (200 ml) and washed with sodium bicarbonate solution and brine (100 ml each). After drying over anhydrous sodium sulfate, the solvent was evaporated to give a residue which was chromatographed to give yellowish oil (2.90 g, 94%).

¹H NMR (400 MHz, CDCl₃), δ=1.96-2.02 (2H, m), 2.11-2.16 (1H, m), 2.21 (3H, s), 3.20 (1H, m), 5.62 (1H, dd, J=2.48, 8.98 Hz), 6.15 (1H, br-s), 7.21 (3H, dd, J=7.29, 7.30 Hz), 7.32 (6H, dd, J=7.33, 8.14 Hz), 7.44 (1H, ddd, J=1.11, 8.36, 8.36 Hz), 7.52 (6H, d, J=7.61 Hz), 7.70 (1H, ddd, J=1.20, 8.32, 8.32 Hz), 7.95 (1H, dd, J=1.30, 8.29 Hz), 8.05 (1H, dd, J=0.78, 7.98 Hz).

### Example 5

### Synthesis of 1-(2-nitrophenyl)-3-(N-methyl-N-tritylamino)propyl 1H-imidazole-1-carboxylate (7)

1-(2-nitrophenyl)-3-(N-methyl-N-tritylamino)propanol (6) (888 mg, 1.96 mmol) was dissolved in dichloromethane (20 ml). 1,1′-Carbonyldiimidazole (1.62 g, 10 mmol) was added to the mixture. The reaction was kept at room temperature for 4 hours. The mixture was diluted with dichloromethane (50 ml) and the solution was washed with water (50 ml) two times and dried over anhydrous sodium sulfate. After removal of the solvent, the residue was purified on a silica gel column to give a yellow solid (770 mg, 72%).

¹H NMR (400 MHz, CDCl₃), δ=2.15 (3H, s), 2.30-2.61 (4H, m), 6.59 (1H, dd, J=2.57, 9.75 Hz), 7.07 (1H, m), 7.14 (3H, m), 7.23 (6H, dd, J=7.18, 8.09 Hz), 7.29 (1H, m), 7.49 (6H, d, J=7.34 Hz), 7.52 (1H, m), 7.61 (1H, dd, J=1.53, 7.93 Hz), 7.68 (1H, ddd, J=1.15, 7.99, 8.78 Hz), 8.00 (1H, s), 8.06 (1H, dd, J=0.92, 8.02 Hz). i) the average of the pKa values of the individual carboxylic acid moieties is in the range of 5.0 to 7.4.

### Example 6

Photo-Morpholinos, developed in part based on experimental results using the “TnT T7 Quick Coupled Transcription/Translation System” from Promega Corporation, where targeted against the “no tail” gene in zebrafish and were found in experiments in zebrafish embryos to exhibit the desired gene modulations.

For the light-on application (see FIG. 4a) the antisense Photo-Morpholino had the following sequence:

5′ AGCTTGAGATTTXAGCGACGATCCT,

where X is the photolinker

For the light-off application (see FIG. 5a) the antisense Morpholino had the following sequence:

5′ AGCTTGAGATAAGTCCGACGATCCT

and the sense Photo-Morpholino had the following sequence:

5′ GATCGTCGGAXTTATCTCAAG.

The “TnT T7 Quick Coupled Transcription/Translation System” from Promega Corporation

1. (canceled)

2. (canceled)

3. (canceled)

4. (canceled)

5. A Photo-Morpholino containing at least one photolinker inserted between Morpholino sequences, selected from the forms: 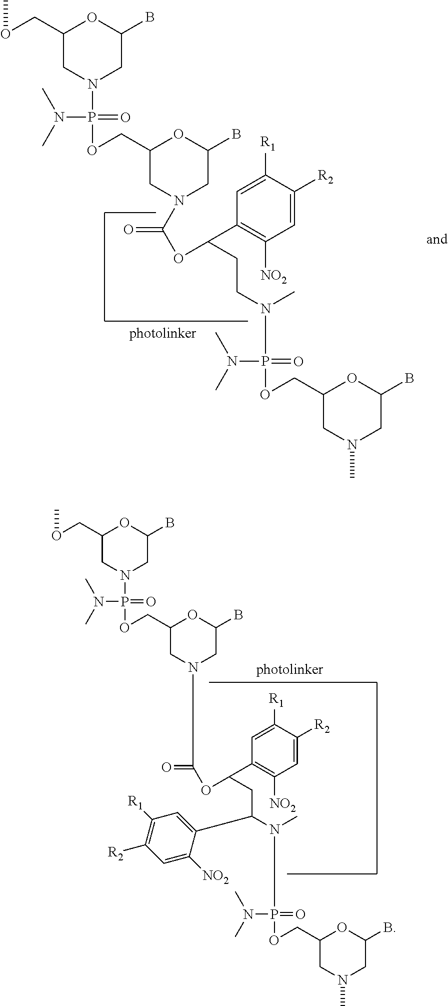
wherein:
an R₁ is selected from the group consisting of hydrogen and methoxy; and
an R₂ is selected from the group consisting of hydrogen and methoxy.

6. The Photo-Morpholino of claim 5, wherein the R₁ is hydrogen and the R₂ is hydrogen.

7. The Photo-Morpholino of claim 5 is used in combination with a complementary Morpholino.

8. The Photo-Morpholino of claim 5, comprising:
at least one photolinker, and
at least 12 Morpholino subunits.

9. The Photo-Morpholino of claim 8, comprising:
two photolinkers, and,
at least 21 Morpholino subunits.

10. A composite light-cleavable structure comprising:
a) a conventional antisense Morpholino;
b) a sense Photo-Morpholino of claim 5
containing one photolinker, and
containing at least 12 Morpholino subunits; and,
c) a photocleavable leash covalently linking the antisense Morpholino and the sense Photo-Morpholino.

11. A photolinker subunit having a structure shown below: 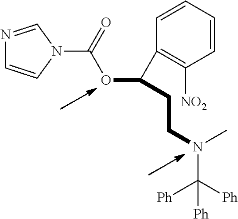
the structure consisting of:
a feature of which a bond distance between oxygen and nitrogen atoms matches that of a Morpholino subunit: 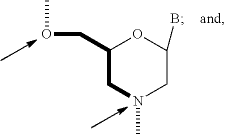
a second feature of which the 1H-imidazole-1-carboxylate is effective to form a covalent bond with a nitrogen atom of the Morpholino subunit under Morpholino assembly conditions; and an amine protected by a trityl group to continue a Morpholino elongation after removal of the trityl group.


SEQUENCE LISTING (5 sequences)

SEQ ID NO: 1 - DNA, 25 bp, Danio rerio
  Morpholino_phosphorodiamidate_oligo_backbone (1)..(25)
agcttgagataagtccgacgatcct

SEQ ID NO: 2 - DNA, 12 bp, Danio rerio
  Morpholino_phosphorodiamidate_oligo_backbone (1)..(12)
  Light-on_photocleavable_group_5′_sequence (12)..(12): This Morpholino oligo sequence is covalently linked at its 3′ end to a photocleavable moiety and that photocleavable group is further linked to the 5′ end of the light-on notail antisense Morpholino 3′ sequence.
agcttgagattt

SEQ ID NO: 3 - DNA, 12 bp, Danio rerio
  Light-on_photocleavable_group_3′_sequence (1)..(1): This Morpholino oligo sequence is covalently linked at its 5′ end to a photocleavable moiety and that photocleavable group is further linked to the 3′ end of the light-on notail antisense Morpholino 5′ sequence.
agcgacgatcct

SEQ ID NO: 4 - DNA, 10 bp, Danio rerio
  Morpholino_phosphorodiamidate_oligo_backbone (1)..(10)
  Light-off_photocleavable_group_5′_sequence (10)..(10): This Morpholino oligo sequence is covalently linked at its 3′ end to a photocleavable moiety and that photocleavable group is further linked to the 5′ end of the light-off notail antisense Morpholino 3′ sequence.
gatcgtcgga

SEQ ID NO: 5 - DNA, 10 bp, Danio rerio
  Light-off_photocleavable_group_3′_sequence (1)..(1): This Morpholino oligo sequence is covalently linked at its 5′ end to a photocleavable moiety and that photocleavable group is further linked to the 3′ end of the light-off notail antisense Morpholino 5′ sequence.
ttatctcaag